(19) United States
(12) Patent Application Publication
(10) Pub. No.: US 2012/0240840 A1
(43) Pub. Date: Sep. 27, 2012

(54) DOG DOOR CHIMES

(76) Inventor: MARK DAVID DEROUCHIE, Alpharetta, GA (US)

(21) Appl. No.: 13/070,696

(22) Filed: Mar. 24, 2011

(51) Int. Cl. G10K 1/00

(52) U.S. Cl. 116/141

(57) ABSTRACT

An alert system allows a dog to ring chimes that has been specially made for dogs, although the alert system can be used for other household pets as well. A beautiful sound resonates throughout the home to inform individuals that the dog needs to go out. The alert system may include a plurality of chimes arranged on a base that is either attached to a door hinge pin or mounted on a wall. Unlike conventional wind chimes, the dog door chimes does not include a wind catcher to cause the chimes to ring. Instead, the chimes are configured to sound when nudged by a dog's paw, body, head or nose, for example. The dog door chimes may include a relatively large ringer to prevent chewing thereupon by dogs.

### BACKGROUND OF THE INVENTION

The present invention relates to door chimes and, more particularly, to a door chime alert system made exclusively for dogs, although the alert system can be used for other household pets as well, which is installed at a home entry door by a hinge or a wall mount.

Currently, dogs scratch on doors and molding at a door entry when they want to go out, which causes damage. At other times, they may bark, which creates excitement and noise. Plus other times, dogs may go to the bathroom in the house, which causes a mess.

As can be seen, there is a need for an alert system that allows a dog to alert a person without damage and without unpleasant noise.

### SUMMARY OF THE INVENTION

In one aspect of the present invention, an alert system comprises a top support member; a plurality of chimes suspended from the top support member; and a mount adapted to secure the top support member to a wall or a hinge.

In another aspect of the present invention, an alert system comprises a top support member; a plurality of chimes suspended from the top support member; a ringer suspended from the top support member; an elongated hinge pin member, adapted to fit inside a hinge; a hinge pin arm attached to and extending away from the elongated hinge pin member; and a hinge pin stabilizing flare adapted to frictionally fit inside an upper segment of the hinge to prevent the hinge pin arm from moving when a door attached by the hinge is opened or dosed

These and other features, aspects and advantages of the present invention will become better understood with reference to the following drawings, description and claims.

### BRIEF DESCRIPTION OF THE DRAWINGS

FIG. 1 is a perspective view of dog door chimes, according to an exemplary embodiment of the present invention;

FIG. 2 is a detailed perspective view taken along line 2-2 of FIG. 1;

FIG. 3 is a side elevation view of a hinge pin mount of the dog door chimes of FIG. 1;

FIG. 4 is a detailed cross-sectional view of a hinge pin mounting assembly of the dog door chimes of FIG. 1;

FIG. 5 is a detailed cross-sectional view taken along line 5-5 of FIG. 2; and

FIG. 6 is a detailed perspective view of wall mounted dog door chimes, according to another exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides an alert system that allows a dog to ring chimes that has been specially made for dogs, although the alert system can be used for other household pets as well. A beautiful sound resonates throughout the home to inform individuals that the dog needs to go out. The alert system may include a plurality of chimes arranged on a base that is either attached to a door hinge pin or mounted on a wall. Unlike conventional wind chimes, the dog door chimes does not include a wind catcher to cause the chimes to ring. Instead, the chimes are configured to sound when nudged by a dog's paw, body, head or nose, for example. The dog door chimes may include a relatively large ringer to prevent chewing thereupon by dogs.

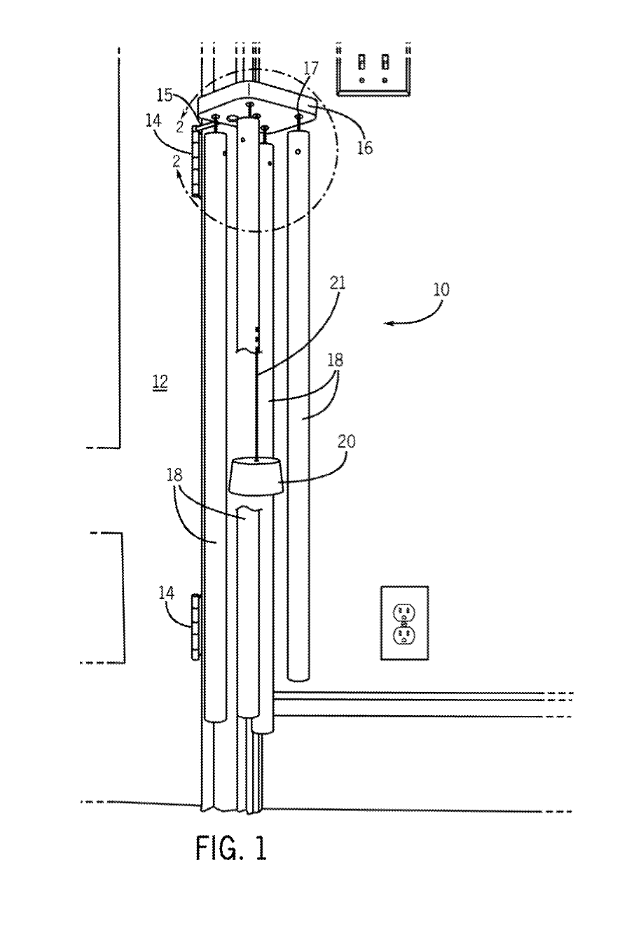

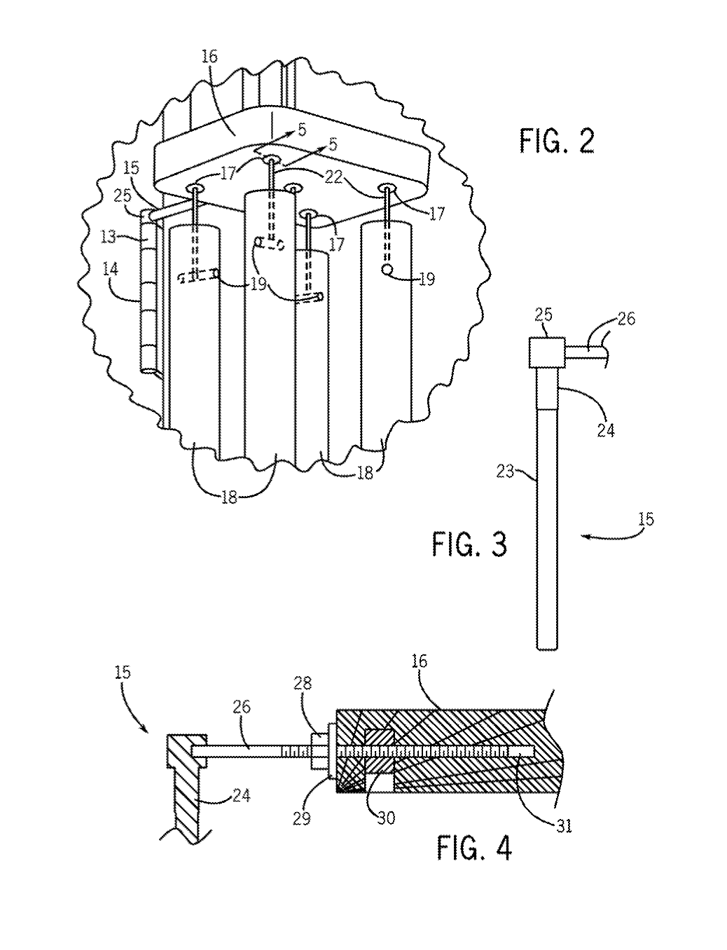

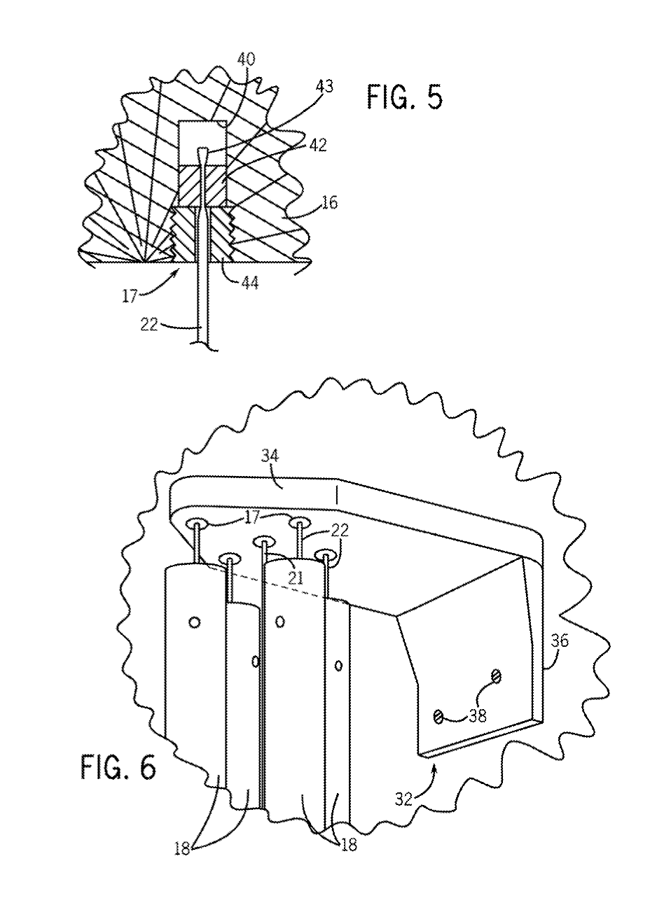

Referring to FIGS. 1 through 5, an alert system 10 (also referred to as dog door chimes 10) may include a plurality of chimes 18 suspended from a top support member 16. Cords 22, as described in greater detail below, may be used to suspend the chimes 18 from the top support member 16. A ringer 20 may be suspended from the top support member 16 by a suspension cord 21. The plurality of chimes 18 may include from 2 to about 8 chimes 18, typically about 4 chimes 18. The chimes 18 may be between about 0.25 inch and about 4 inches in diameter, typically about 1.25 inches in diameter. The chimes 18 may be between about 5 inches to about 40 inches in length, typically between 31 inches and 37 inches in length. The ringer 20 may be centrally disposed between the suspended chimes 18. The top support member 16 may be from about 0.02 inch to about 4 inches thick, typically about (1 inch thick and may have a length and width (or diameter) from about 2 inches to about 10 inches, typically about 5.5 inches. The top support member 16 may take the form of various shapes, including square, rectangular, circular, oval and the like.

In some embodiments, the alert system 10 may be attached to a hinge 14 of a door 12 with a specially designed hinge pin 15. The hinge pin 15, as shown in FIG. 3, may include a hinge an elongated hinge pin member 23 having a hinge pin stabilizing flare 24 and a hinge pin cap 25 at one end thereof. A hinge pin arm 26 may extend outwardly from the hinge pin cap 25. Typically, the hinge pin arm 26 will extend orthogonally outward from the hinge pin cap 24.

The diameter of the hinge pin stabilizing flare 24 may be sized to frictionally fit into an upper segment 13 of the hinge 14. The hinge pin stabilizing flare 24 may prevent the hinge pin from moving when the door 12 opens or closes. The hinge pin cap 25 may have a diameter roughly the same as the diameter of the upper segment 13 of the hinge 14. The hinge pin cap 25 may prevent the hinge pin 15 from going too far down into the hinge 14 and may also provide a clean transition to the hinge pin arm 26.

As shown in FIG. 4, the hinge pin arm 26 may be threaded and a nut 28 and washer 29 may be disposed on the threaded arm. The top support member 16 may have a hole 31 formed therein for accepting the hinge pin arm 26.

A dowel nut 30 may be disposed in the top support member 16, coaxially with the hole 31. The threaded end of the hinge pin arm 26, having the nut 28 and washer 29 already threaded thereupon, may be inserted into the hole 31 and threaded into the dowel nut 30. The user may adjust the position of the top support member 16 relative to the hinge 14 by threading or unthreading the hinge pin arm 26 from the dowel nut 30. Once a desired length is achieved, the nut 28 may be tightened against a side of the top support member 16 to secure the top support member 16 to the hinge pin arm 26.

The chimes 18 may be attached to the top support member 16 via the cords 22. Spring pins 19 may be used to secure one end of the cords 22 to the chimes 18. A bore 40 may be formed in the top support member 16. A cord attachment 17 may be formed within the bore 40. The cord attachment 17 may include a wood insert 44 and a stop/button sleeve 42. The wood insert 44, having a central hole formed therethrough, may frictionally fit (or may thread) into the bore 40. The stop/button sleeve 42 may be disposed inside the bore 40, held in place by the wood insert 44. The stop/button sleeve 42 may have a central hole formed therethrough that aligns with the central hole of the wood insert 44. The cord 21, 22, may pass through both central holes and may be formed with a flared end 43 to keep the cord 21, 22 from passing out of the central hole of the stop/button sleeve 42.

Referring now to FIG. 6, a wall mount 32 may include a mounting plate 36 for attaching to a wall via, for example, mounting screws 38. The mounting plate 36 may have a top support member 34 attached thereto. The top support member 34 may have a design similar to top support member 16, except that the top support member 34 may be suitably shaped to support the chimes 18 as a desired distance away from the wall.

It should be understood, of course, that the foregoing relates to exemplary embodiments of the invention and that modifications may be made without departing from the spirit and scope of the invention as set forth in the following claims.

What is claimed is:

1. An alert system comprising:
a top support member;
a plurality of chimes suspended from the top support member; and
a mount adapted to secure the top support member to a wall or a hinge.

2. The alert system of claim 1, further comprising a ringer suspended from the top support member.

3. The alert system of claim 1, wherein the ringer is centrally disposed between the plurality of chimes.

4. The alert system of claim 1, wherein the top support member is attached to a hinge via a hinge pin.

5. The alert system of claim 4, wherein the hinge pin includes an elongated hinge pin member, adapted to fit inside the hinge, and a hinge pin arm extending away from the elongated hinge pin member.

6. The alert system of claim 5, wherein the elongated hinge pin member includes a hinge pin stabilizing flare adapted to frictionally fit inside an upper segment of the hinge.

7. The alert system of claim 5, wherein the hinge pin arm includes a threaded portion adapted to thread into threads in the top support member.

8. The alert system of claim 1, wherein the mount is a wall mount bracket.

9. An alert system comprising:
a top support member;
a plurality of chimes suspended from the top support member;
a ringer suspended from the top support member;
an elongated hinge pin member, adapted to fit inside a hinge;
a hinge pin arm attached to and extending away from the elongated hinge pin member; and
a hinge pin stabilizing flare adapted to frictionally fit inside an upper segment of the hinge to prevent the hinge pin arm from moving when a door attached by the hinge is opened or closed.

10. The alert system of claim 9, wherein the hinge pin arm includes a threaded portion adapted to thread into threads in the top support member.
